(19) United States
(12) Patent Application Publication
(10) Pub. No.: US 2022/0211321 A1
(43) Pub. Date: Jul. 7, 2022

(54) LIMB MOTION TRACKING BIOFEEDBACK PLATFORM AND METHOD OF REHABILITATION THERAPY FOR PATIENTS WITH SPASTICITY

(71) Applicants: THE TRUSTEES OF COLUMBIA UNIVERSITY IN THE CITY OF NEW YORK, New York, NY (US); TEACHERS COLLEGE, New York, NY (US)

(72) Inventors: Blynn L. SHIDELER, Gibsonia, PA (US); Maxime ROBERT, Lac Beauport (CA); Elizabeth Anne SHROUT, Potomac, MD (US); Katherine Claire CAVANAUGH, Chesterfield, MO (US); Rachel ALEXANDER, Silver Spring, MD (US)

(73) Assignees: THE TRUSTEES OF COLUMBIA UNIVERSITY IN THE CITY OF NEW YORK, New York, NY (US); TEACHERS COLLEGE, New York, NY (US)

(21) Appl. No.: 17/701,919

(22) Filed: Mar. 23, 2022

Related U.S. Application Data
(63) Continuation of application No. PCT/US2020/047607, filed on Aug. 24, 2020.
(60) Provisional application No. 62/890,265, filed on Aug. 22, 2019.

(51) Int. Cl. A61B 5/00; A61B 5/11; G16H 20/30; G16H 40/67

(52) U.S. Cl. CPC A61B 5/486; A61B 5/1114; A61B 5/743; A61B 5/1123; A61B 5/7207; A61B 5/4082; A61B 2503/06; G16H 20/30; G16H 40/67; A61B 2505/09; A61B 2562/0219; A61B 2562/0223; A61B 5/681

(57) ABSTRACT

A limb tracking biofeedback platform and rehabilitation therapy methodology for spasticity patients improves and maintains limb functionality permitting compliance monitoring, patient encouragement and biofeedback outside the presence of a therapist. The method includes defining a desired therapeutic limb motion, and a goal for repetitions of the therapeutic motion over a given period. The platform includes at least one sensor coupled to the patient's limb which is configured to track at least one of limb movement and orientation. Tracked movement and/or orientation data is transmitted to a platform controller which calculates a number of therapeutic movements of the patient's limb based upon the tracked movement and/or orientation data. Biofeedback to the patient is provided by displaying at least the number of therapeutic movements. Typically reminder prompts and positive reinforcement of goals achieved are also displayed to the patient. The therapist can also have access to the recorded data.

### CROSS-REFERENCE TO RELATED APPLICATION

This application is a continuation of International Patent Application No. PCT/US2020/047607 filed Aug. 24, 2020, which claims priority to U.S. Provisional Patent Application No. 62/890,265, which was filed on Aug. 22, 2019, the entire contents of which are incorporated by reference herein.

### FIELD

The present subject matter relates to a limb motion tracking biofeedback platform and associated method of rehabilitation therapy for patients with spasticity, more specifically to an upper limb motion tracking biofeedback platform and associated therapy for children with hemiplegic spastic cerebral palsy or hemiparetic stroke.

### BACKGROUND

Cerebral palsy (CP) is a neuromuscular disorder that affects functional movement, posture, and balance. Cerebral palsy is a common childhood motor disability, affecting 1 in 323 children in America and 17 million people worldwide. A significant amount of cerebral palsy patients suffer from hemiplegic, spastic cerebral palsy. Hemiplegia and spasticity are common symptoms of other pediatric movement disorders, such as perinatal stroke, which affects 1 in every 2800 births. The challenges that children with hemiplegia face are performing tasks requiring bilateral manipulation, and as these children develop, they tend to acquire ever greater skill with the unaffected hand and increasingly neglect the impaired hand. Additionally, impaired hand neglect in young CP and stroke patients can lead to psychosocial clinical consequences that include reduced self-confidence and higher dependence on others.

Certain upper limb assistive robotics have been developed for cerebral palsy and other neuromuscular disorders. For example, a certain upper limb powered robotic exoskeleton has been developed, and a certain upper limb robot with associated therapy is also developed. These robotic solutions are not practical for daily use, are relatively expensive, have a high rate of abandonment, and are often beneficial only for most-severely-affected individuals.

Currently, the common method to help improve range of motion and muscle strength in the affected arm of hemiplegic patients is with Constraint-Induced Movement Therapy (CIMT), which entails the restraint of the unaffected arm, usually with a sling or cast, and encouraging them to use their affected arm to perform tasks. Bilateral movement therapies such as Hand-Arm Bilateral Intensive Therapy (HABIT) for CP patients have shown that the efficacy of hand rehabilitation is not dependent on the use of restrictive devices on the unaffected hand. Despite efforts of physical and occupational therapists (collectively referenced herein as therapists or clinicians) in clinics, hemiplegic patients tend to regress to using the unaffected hand in the absence of clinicians, and the effect of the newly learned functions from CIMT fades away without proper practice and reminding.

It is beneficial to provide a simple device to encourage children with hemiplegic spastic cerebral palsy to actively use their affected upper limb in the absence of a clinician or therapist.

### SUMMARY

It is an object of the presently disclosed subject matter to provide an effective and efficient limb tracking biofeedback platform and method of motion rehabilitation therapy for patients with spasticity, which improves and maintains limb functionality and permits compliance monitoring, patient encouragement and patient biofeedback outside the presence of a therapist. The method includes defining rehabilitation therapy for a patient including a desired therapeutic motion for a limb of the patient, and a goal for the number of repetitions of the therapeutic motion over a given time period. The limb tracking biofeedback platform includes at least one sensor that is coupled to the limb of the patient, wherein the sensor is configured to track at least one of movement and orientation of the limb. The platform includes a controller wherein the tracked movement and/or orientation data is transmitted to a controller which calculates a number of therapeutic movements of the limb of the patient based upon the tracked movement and/or orientation data. The limb tracking biofeedback platform provides biofeedback to the patient by displaying at least the number of therapeutic movements to the patient, and typically reminder prompts and positive reinforcement of goals achieved are also displayed to the patient. The therapist can also have access to the recorded tracked results of the number of therapeutic motion.

In certain embodiments, the disclosed subject matter provides a limb motion tracking biofeedback platform including a plurality of sensors configured to be coupled to a limb of a patient. The plurality of sensors also configured to track and transmit movement and orientation of the limb. The limb motion tracking biofeedback platform further includes a controller configured to receive the tracked movement and orientation data of the limb of the patient. The controller is also configured to calculate a number of predetermined therapeutic movements of the limb based upon the tracked movement and orientation data. The limb motion tracking biofeedback platform further includes a display configured to display at least the number of therapeutic movements of the limb to the subject.

In certain embodiments, the disclosed sensors comprise a magnetometer, an accelerometer, a gyroscope, or combinations thereof. In non-limiting embodiments, the magnetometer is configured to generate data for distinguishing incidental and therapeutic motion. In some embodiment, the sensors are configured to be coupled to a wrist of the subject.

In certain embodiments, the limp of the patient comprises a spastic upper limb. In non-limiting embodiments, the patient is a pediatric patient. In some embodiments, the tracked movement and/or orientation data comprises a waveform data measured from the subject.

In certain embodiment, the disclosed subject matter provides a method of motion tracking, biofeedback based, limb rehabilitation therapy for patients with spasticity including defining rehabilitation therapy for a patient including a desired therapeutic motion for a limb of the patient; coupling at least one sensor to the limb of the patient wherein the sensor is configured to track at least one of movement and orientation of the limb; transmitting the tracked movement and/or orientation data to a controller; calculating a number of therapeutic movements of the limb of the patient based upon the tracked movement and/or orientation data; displaying at least the number of therapeutic movements to the patient.

The method of rehabilitation therapy according to one embodiment of the disclosed subject matter can provide wherein the limb is an upper limb of the patient, and the sensors are coupled to the wrist of the patient. One embodiment can further include displaying at least the number of therapeutic movements to the patient.

The method of rehabilitation therapy according to one embodiment of the disclosed subject matter can provide for further including setting at least one goal for the patient and providing positive reinforcement to the patient upon attaining a preset goal, and further including maintaining a historical record of therapy by the patient, and further including providing encouragement to the patient when falling behind a preset goal.

The method of rehabilitation therapy according to one embodiment of the disclosed subject matter can further include graphically displaying the data to the patient. The method of rehabilitation therapy according to one embodiment of the disclosed subject matter can provide wherein the sensors include a magnetometer, an accelerometer, and a gyroscope, and wherein calculating a number of therapeutic movements of the limb of the patient based upon the tracked movement and/or orientation data includes waveform recognition, and further wherein the data obtained from the magnetometer is utilized to distinguish between incidental and therapeutic motion.

The method of rehabilitation therapy according to one embodiment of the disclosed subject matter can further include maintaining a record of clinician review of the data, and wherein tracked movement and/or orientation data and the calculated number of therapeutic movements are stored online.

In certain embodiments, the disclosed subject matter provides a method of motion tracking, biofeedback based, upper limb rehabilitation therapy for a pediatric patient with hemiplegic spastic cerebral palsy including defining rehabilitation therapy for a patient including a desired therapeutic motion for a spastic upper limb of the pediatric patient; coupling a plurality of sensors to the spastic upper limb of the pediatric patient wherein the sensors are configured to track movement and orientation of the spastic upper limb;

transmitting the tracked movement and orientation data of the spastic upper limb of the pediatric patient to a controller; calculating a number of therapeutic movements of the spastic upper limb of the patient based upon the tracked movement and orientation data, and displaying at least the number of therapeutic movements of the spastic upper limb to the patient.

These and other advantages of the presently disclosed subject matter will be clarified in the brief description of the preferred embodiment.

### REFERENCE NUMERAL LISTING

The following reference numerals are used in the description of the preferred embodiments and in the figures in which like reference numerals represent like elements throughout:

10—Motion Tracking Biofeedback Platform or Platform.

20—Patient.

22—Spastic upper limb of the pediatric patient.

24—Unaffected upper limb of the pediatric patient.

30—Therapist or clinician.

40—Cloud.

100—Wrist mounted sub-assembly of platform.

110—Housing of sub-assembly 100.

120—Adjustable mounting straps of sub-assembly 100.

130—IMU-sensors of sub-assembly 100.

140—Transmission Unit of sub-assembly 100.

150—Data storage of subassembly 100.

160—Power supply of subassembly 100.

170—Display.

200—Controller.

210—Computer.

300—Smartphone/PDA.

1000—Method of motion tracking, biofeedback based, upper limb rehabilitation therapy for a pediatric patient with hemiplegic spastic cerebral palsy.

1010—Method of defining rehabilitation therapy for a patient including a desired therapeutic motion for a spastic upper limb of the pediatric patient.

1020—Method of coupling a plurality of sensors to the spastic upper limb of the pediatric patient wherein the sensors are configured to track movement and orientation of the spastic upper limb.

1030—Method of transmitting the tracked movement and orientation data of the spastic upper limb of the pediatric patient to a controller.

1040—Method of calculating a number of therapeutic movements of the spastic upper limb of the patient based upon the tracked movement and orientation data.

1050—Method of displaying at least the number of therapeutic movements of the spastic upper limb to the patient and/or the therapist.

### BRIEF DESCRIPTION OF THE DRAWINGS

FIG. 1 is a schematic flowchart illustration of a method of motion tracking, biofeedback based, upper limb rehabilitation therapy for a pediatric patient with hemiplegic spastic cerebral palsy according to one embodiment of the presently disclosed subject matter.

FIG. 2 is a schematic view of an upper limb motion tracking biofeedback platform for children with hemiplegic spastic cerebral palsy according to one embodiment of the presently disclosed subject matter.

FIGS. 3A-3C are schematic views of possible representative feedback to the patients on a wearable subassembly of the upper limb motion tracking biofeedback platform according to FIG. 2.

FIGS. 4A and 4B are schematic views of possible representative feedback to the patients on a separate smartphone for reach based therapeutic motion of the upper limb motion tracking biofeedback platform according to FIG. 2.

FIG. 5 is a schematic illustration of the calculation of reach count, velocity, and distance metrics for reach based therapeutic motion of the upper limb motion tracking biofeedback platform for children with hemiplegic spastic cerebral palsy according to FIG. 2.

FIGS. 6A and 6B are schematic views of patient movements during a reach based therapeutic motion.

FIGS. 7A and 7B are schematic views of patient movements during ambulatory motion without a reach based therapeutic motion and patient movements during ambulatory motion with a reach based therapeutic motion, respectively.

FIG. 8 is a schematic chart of a process for calculating reach count for reach based therapeutic motion of the upper limb motion tracking biofeedback platform for children with hemiplegic spastic cerebral palsy according to FIG. 2.

It is to be understood that both the foregoing general description and the following detailed description are exemplary and are intended to provide further explanation of the disclosed subject matter.

### DETAILED DESCRIPTION

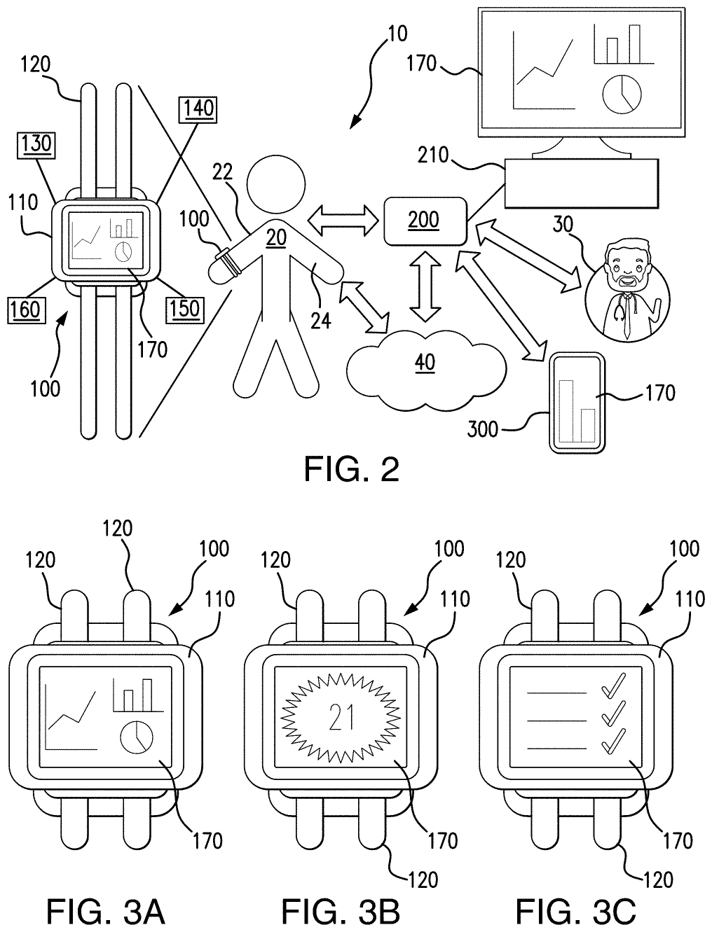

The present subject matter is directed, in one preferred embodiment, to an upper limb motion tracking biofeedback platform 10 and associated therapy for children or patients 20 with hemiplegic spastic cerebral palsy. FIG. 2 is a schematic view of an upper limb motion tracking biofeedback platform 10 for children or patients 20 with hemiplegic spastic cerebral palsy according to one embodiment of the present subject matter. The limb motion tracking biofeedback platform 10 including a plurality of sensors 130 configured to be coupled to the spastic upper limb 22 of the pediatric patient 20 wherein the sensors 130 are configured to track movement and orientation of the spastic upper limb 22 and wherein the plurality of sensors 130 include a magnetometer, an accelerometer and a gyroscope; a controller 200 wherein the controller is configured to receive the transmitted tracked movement and orientation data of the spastic upper limb 22 of the pediatric patient 20; and wherein the controller 200 is configured to calculate a number of therapeutic movements of the spastic upper limb 22 of the patient 20 based upon the tracked movement and orientation data; and a display 170 configured for displaying at least the number of therapeutic movements of the spastic upper limb 22 to the patient 20 and therapist 30. Therapist and clinician (reference numeral 30) are used interchangeably herein in relation to the platform 10, although in practice these titles can reflect distinctly different roles performed by distinct individuals.

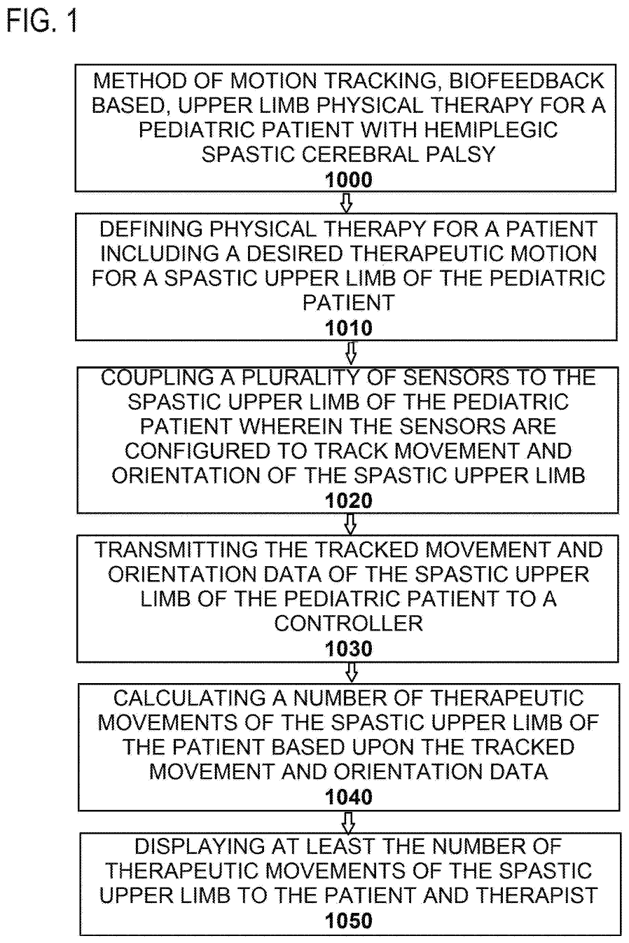

FIG. 1 is a schematic flowchart illustration of a method 1000 of motion tracking, biofeedback based, upper limb rehabilitation therapy for a pediatric patient 20 with hemiplegic spastic cerebral palsy according to one preferred embodiment of the present subject matter. The method 1000 includes 1010 of the clinician or therapist 30 defining rehabilitation therapy for a patient 20, which includes a desired therapeutic motion for a spastic upper limb 22 of the pediatric patient 20. The method 1000 includes 1020 of coupling a plurality of sensors 130 to the spastic upper limb 22 of the pediatric patient 20 wherein the sensors 130 are configured to track movement and orientation of the spastic upper limb 22 of the patient 20. The method 1000 includes 1030 of transmitting the tracked movement and orientation data of the spastic upper limb 22 of the pediatric patient 20 to a controller 200. The method 1000 includes 1040 of calculating a number of therapeutic movements of the spastic upper limb 22 of the patient 20 based upon the tracked movement and orientation data. The method 1000 includes 1050 of displaying via one or more displays 170 at least the number of therapeutic movements of the spastic upper limb 22 to the patient 20 and/or the therapist 30.

### Wrist Mounted Sub-Assembly 100

A cornerstone of the platform 10 of the present subject matter is the wearable subassembly 100 or the wrist mounted sub-assembly 100 of platform 10. The subassembly 100 can be referenced as a bracelet or wearable device or wearable component within this application. The platform 10 provides a sub-assembly 100 for coupling to the patient 20 on the affected limb 22.

As discussed above, therapists and clinicians 30 in the field have identified a need for a device like platform 10 that reminds children or patients 20 with cerebral palsy to use their affected arm 22 in everyday life since the patients 20 often regress to using their less affected arm 24. The platform 10 with bracelet 100 addresses this problem can lead to the maintenance of arm functionality in the affected arm 22 that children 20 gain during clinical therapy sessions, which can lead to an improved quality of life for these patients 20. The platform 10 provides a solution, called BUDI by the developers, in the form of the wearable bracelet 100.

It is an anticipated alternative that the subject matter can further include a second subassembly or bracelet 100 for the unaffected limb 24 for collecting relative data of the usage of the limbs 22 and 24. The platform 10, as described herein, works effectively and efficiently with a single sub-assembly 100 for the affected limb 22, and the use of a single bracelet 100 significantly reduces the costs (as opposed to two sub-assemblies 100), and a reduction in costs can yield higher patient implementation and thus better overall results.

The wrist mounted sub-assembly 100 or bracelet includes a housing 110 and an adjustable user attachable strap 120 that operates like a conventional watch band and allows the subassembly 100 to be used with a wide range or size of children 20.

The housing 110 is equipped with sensor 130 in the form of an Inertial Measurement Unit (IMU) 130, transmitting unit 140, data storage unit 150, power supply 160, and display 170, as discussed below. The fact that the subassembly 100 looks like, and is sized similar to, and is coupled to the user in a manner similar to, a watch is an intentional feature of the subject matter to encourage children 20 to want to wear the subassembly 100 to promote the use and acceptance of the platform 10 by the patients 20. As the subassembly 100 looks like a watch and it doesn't get in the way of normal activities, nor does it draw undesired attention, the patients 20 can be more inclined to wear it, as opposed to something that is obviously specialized medical equipment and or more cumbersome.

### Sensors/IMU 130

The sensors 130 are in the form of an IMU 130, which is a combination of an accelerometer, a gyroscope, a magnetometer, which can sense multiple degrees of freedom. For further details on IMU. IMU devices are well known for general motion tracking. In the context of bracelet 100, the IMU 130 is useful for motion tracking of the limb 22 as it is easily and reliably able to detect arm movement in any direction, allowing for accurate measurement of the amount of arm usage.

The IMU 130 for the bracelet 100 is a combination of three components: an accelerometer, a gyroscope, and a magnetometer. It is an electronic device that uses these components to measure and report a body's specific force, angular rate, and the magnetic field surrounding the body. An accelerometer is an electronic device that measures acceleration indirectly through a force that is applied to one of its walls. This force is an inertial force that occurs in the opposite direction of the acceleration vector. The accelerometer quantifies the magnitude of the force and the direction of this force in the x-y-z plane. A gyroscope measures the rate of changes of different angles around the x-y-z-axes (e.g., in deg/sec). It outputs a value that is linearly related to the rate of change of these angles. In IMU 130, the gyroscope quantifies the rotation of the device in space. A magnetometer is an electrical device that measures the strength and direction of magnetic fields around a body. In essence, it is a digital compass. It measures the device's orientation in the x-y-z plane of the earth's magnetic fields via rotation and tilt.

The IMU 130 combines all three electronic devices or sensors by aligning the coordinate systems. Each component can be used to smooth out the errors or sensitivity issues of the others, creating a robust measurement system. The platform 10 has been successfully implemented with a SPARKFUN 9DOF RAZOR brand IMU forming IMU 130, which has an Arduino-compatible microprocessor with preloaded firmware that converts the raw analog data to digital data. This IMU 130 can be used to stores data in a micro-SD card forming data storage unit 150, which the user (Patient 20, therapist 30, or caregiver) can be able to remove from the bracelet 100 and place in a computer for data visualization.

Certain techniques can use the raw accelerometer, gyroscope, and magnetometer data from multiple IMUs in several ways to extract meaningful data to quantify functional arm activity in various ways. These techniques can provide arm position data, but they require multiple IMU devices for implementation, increasing both the system cost and computational requirements. Certain comprehensive methods of quantifying upper limb usage through IMUs can be through the kinematic reconstruction of a human arm chain using inverse kinematics. Certain methods model the upper limbs system as a double-inverted pendulum with five degrees of freedom. These methods require using two IMUs, one on each arm segment. Using inverse kinematics “through the use of the pseudo-inverse of the Jacobian,” this method of arm quantification can reconstruct a full arm model that provides local position and angular rotation of all three upper limb joints: the shoulder, the elbow, and the wrist. The accuracy of these estimates has been verified through 3D motion tracking camera technology. However, research on the use of such modeling schemes in the therapeutic setting has concluded that multiple-sensor systems like the inverse kinematic model often create confusion in users, require more processing time than a real-time system can allow, and have poor patient compliance due to setup time. For these various reasons, although these known techniques can be implemented into the platform 10, the preferred method for the platform 10 of the subject matter is obtaining kinematics from a single IMU 130, and the use of this is discussed further below in the calculation of reaches.

### Transmitting Unit 140

The data is transmitted in real time to a controller 200. As discussed below, the controller 200 can be incorporated into the housing 110 and be onboard, although it is more likely to be part of a computer or smartphone 300 associated with the system. Any conventional transmission system can be used, such as a Bluetooth transmission protocol. Bluetooth is a very well-known wireless technology standard for exchanging data between fixed and mobile devices over short distances using short-wavelength UHF radio waves in the industrial, scientific and medical radio bands, from 2.400 to 2.485 GHz.

### Data Storage Unit 150

The data from the IMU 130 can be stored in the removable data storage unit 150, such as a removable micro-SD card, which can be connected to a controller 200 (part of a computer) to then look at the data. For real time applications in which data is transmitted in real time, there can be less need of large data storage in the storage unit 150 or of making the unit 150 removable. The data is stored in a memory unit 150 at least until transmission by unit 140 to the controller 200 for processing. The platform 10 can store historical data at the controller 200 or at a separate location online via the cloud 40.

### Power Supply 160

The bracelet 100 can have an onboard power supply, namely a battery and it can have a battery life that lasts up to 18 hours, weighs less than 48 g (not including the band) have dimensions of at the most 40×38×12 mm (based also on the dimensions of the IMU 130). As a comparison, the heaviest APPLE® watch weights about 48 g (not including the band) and has dimensions of 40×34×10.7 mm, and has a battery life of up to 18 hours. The success of this product with adults and children is believed to establish the acceptability of these design parameters for the bracelet 100 to be easily adopted by patients 20.

The power supply 160 can be rechargeable, and the housing can have an appropriate port for recharging. Further, an on-off switch can be provided on the housing 110.

### Display 170

The platform 10 can be considered to also provide a data visualization platform in the form of visualizing the data and the therapeutic reaches. The visualization can be by way of displays 170 that can be in the housing 110 of the bracelet 100, on a computer coupled to the controller 200 or on separate smartphone or PDA devices 300. The display 170 on the housing 110 can have more abbreviated displays due to a more restricted size.

In one example, the removable data storage unit 150 can be able to connect to a computer and can show daily and weekly arm use of the patient 20, however, real time transmission of data can be one of the preferred methods of implementation. The clinician 30, or even the patient 20, can also be able to set goals for how much use of the affected arm 22 (either alone or as compared to their unaffected arm 24). This data visualization platform allows the patient 20 to better understand their arm 22 usage. Additionally, this information can be accessed by caregivers and/or clinicians 30 (on their own devices 300), allowing them to understand the progress of the child 20.

The platform 10 is thus in one sense a data visualization platform, which can allow patients 20 to see their improvements over time and possibly interact with a wider community of other children 20 with cerebral palsy via a network of patients 20. It is common to have therapy clinics that have a number of patients 20 with similar ages and similar conditions and physical challenges. Thus it is not unexpected that several patients 20 can have similar goals in implementing the platform 10, and sharing results (if deemed appropriate by clinicians 30 and caregivers) between select patients 20 can be viewed as a way of mutual encouragement between patients 20. This aspect can be included with social networking platforms to facilitate communication, if appropriate.

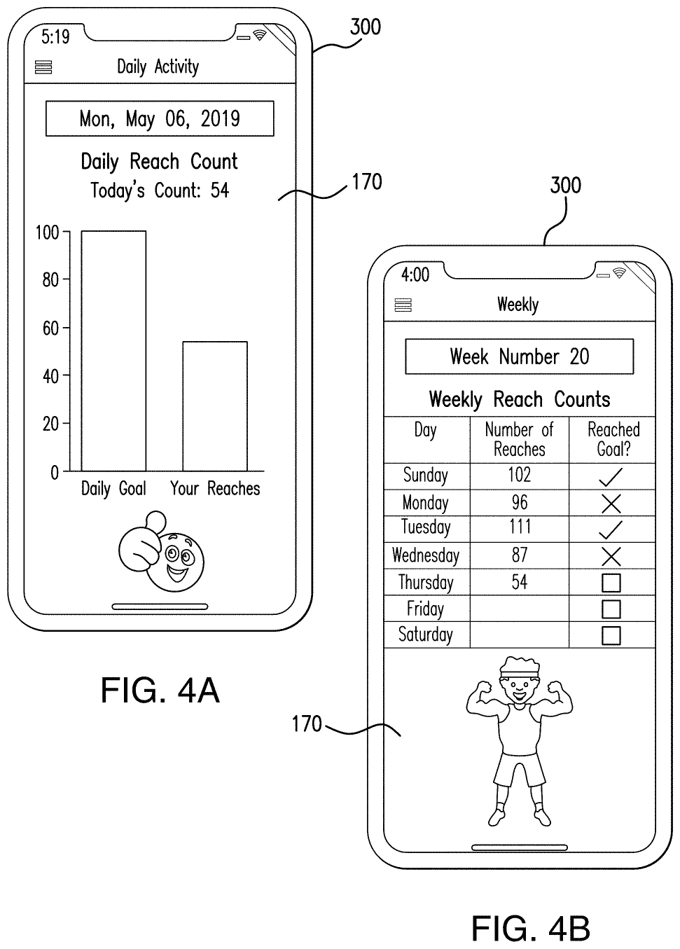

The bracelet 100 can send data via transmission unit 140 to a controller 200, then possibly to a phone application on a device 300. The platform 10 can display data from the day and the week in a way that is easy for children/patients 20 to understand. FIGS. 3A-3C are schematic views of possible representative feedbacks to the patients 20 on the display 170 of the wearable subassembly 100 of the upper limb motion tracking biofeedback platform 10 according to the present subject matter. FIGS. 4A and 4B are schematic views of possible representative feedback to the patients 20 on the display 170 of smartphone device 300 for reach based therapeutic motion of the upper limb motion tracking biofeedback platform 10 according to FIG. 2. Additionally, the child 20 can have the option to have “Friends” that they can compete with while reaching their goals, if the therapists 30 and parental caregivers permit. Motivation to use the affected arm 22 can stem from these interactions in combination with the OLED screen forming the display 170 of the bracelet 100 and the reminder system on the bracelet 100.

### Controller 200

The controller or processor of the platform 10 is shown separately from the bracelet 100 as it need not be included therein. The controller 200 can be part of a separate computer (desktop or laptop or tablet), as shown in FIG. 2 that is effectively running the platform 10 and associated method 1000. The present platform 10 can be implemented largely as a downloadable phone or tablet application such that the controller 200 can alternatively be integral to a device 300. The controller 200 can alternatively be incorporated into the bracelet 100, but that can needlessly increase the cost of the wearable component 100 of the platform 10. The controller 200 can alternatively be maintained by the vendor of the platform 10 and accessible via the cloud 40.

### Smartphone/PDA 300

As noted above, the present platform 10 can be implemented largely as a downloadable phone or tablet application and thus be accessible and displayable on one or more devices 300. The clinicians 30 can have access to the data of each of their patients 20 using the platform 10, and such access is likely through their own device 300. The patient 20 can also have access to the results via their own smartphone device 300 as the larger display can be more useful for certain select display of information. Parental caregivers can also have separate access via their own devices 300. The displays and controls of each device 300 can vary depending upon the participant (i.e., patient, caregiver, or therapist).

Therapist 30 Interaction with Platform 10

The therapist 30 is intended to interact with the platform 10. One aspect of the subject matter can have the platform 10 maintaining a record of clinician review of the data. This can be useful for verification of billing of the clinician 30. The clinician's interface with the platform 10 can integrate with billing software and patient records such that the review and clinician notes are recorded. The clinician notes can include notes for the file/patient record (e.g., “Patient using affected limb sufficiently to avoid loss of limb function gained in therapy, but there is a slight decrease as the time increases from the last sessions”), notes that are to be sent to the patient caregiver (e.g., “Jimmy is doing well with at-home arm usage, but one may want to encourage constant usage the entire time between sessions as there is some drop off as time passes”), and notes that are to be sent to the patient (e.g., “Jimmy, fantastic job! Let's work to keep the daily totals constant for each day. Keep up the great work”).

### 1010 Defining Therapy

The Method 1000 of the present subject matter includes 1010 “Defining Therapy.” This can be described broadly as having the clinician 30 interact with the platform to define rehabilitation therapy for a patient including a desired therapeutic motion for a limb 22 of the patient 20 and a setting at least one goal for the number of desired therapeutic motions for a limb 22 of the patient 20 for the patient 20 over a given time (e.g., per day, per week) and the platform 10 can provide positive reinforcement to the patient 20 upon attaining a preset goal as well as reminders if the patient 20 is falling behind on attaining a goal. The goal can be a range of goals set by the clinician 30 such that the patient 20 (or caregiver) can also set goals (that are within that broader clinician set range) on the platform 10 to better achieve the desired results.

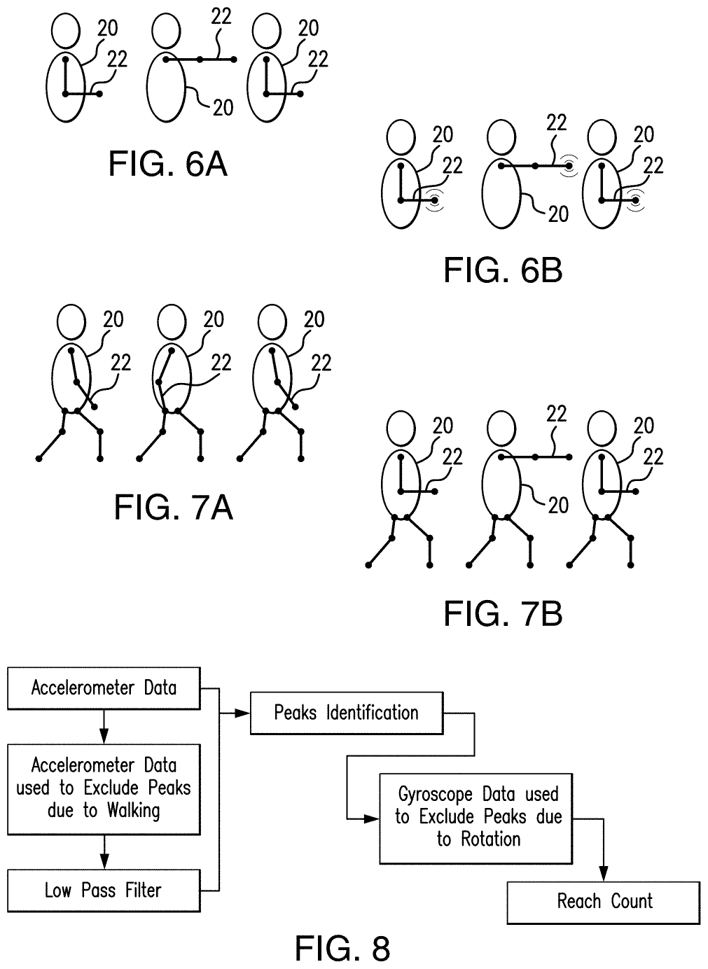

The goal for a given therapeutic motion requires the platform 10 to know and be able to count such motions. The platform 10 of the present subject matter is described relating to a forward reach based therapeutic motion, and FIGS. 6A and 6B are schematic views of patient movements during a forward reach based therapeutic motion. This movement was selected because of its common use in therapy because one of the main symptoms of hemiplegic spastic cerebral palsy (HSCP) is abnormal elbow and wrist contracture. FIG. 6A models the conventional forward reach based therapeutic motions, while FIG. 6B models such a movement incorporating “shaking” of the hand or limb during the motion. A conventional forward reach based therapeutic motion is defined as an action performed within an elevation of ±30 degrees from the horizontal plane. The platform 10 can accurately identify each such motion (within the meaning of this application accurately is defined as greater than 85% correct in counting a therapeutic motion). Illustrating how the platform 10 can be programmed relative to counting a valid therapeutic forward reach, FIGS. 7A and 7B are schematic views of patient movements during ambulatory motion without a reach based therapeutic motion and patient movements during ambulatory motion with a reach based therapeutic motion, respectively. The system needs to be able to distinguish from the conventional motion of the limb 22 from the desired therapeutic motion, which can be, as shown in FIG. 7B, occurring with ambulatory or other patient motions. For the forward reach as the desired motion, the accelerometer data is primarily used to begin a count of reach using peak and/or waveform detection and the magnetometer and gyroscope data is used to generate exclusion criteria to eliminate “false positives” from the accelerometer data.

It is envisioned that the platform 10 can have a plurality of therapeutic motions such as forward reach as shown, vertical reach (where the patient 20 is reaching substantially above the person's head), large arm circles (e.g., where the patient is extending the limb 22 forward and rotating the shoulder through 360 degrees of rotation in what is called a windmill fashion), small arm circles (e.g., where the patient is extending the limb 22 horizontally to the side and rotating the limb to follow small circles of less than +/−30 degrees from horizontal). The platform 10 can provide the clinician 30 with a pull-down menu of known therapeutic motions (i.e., those known to the platform 10) to allow the clinician 30 to select the desired motion(s) and the appropriate goals therefore specific to the needs of a given patient 20.

### 1020 Coupling

The method 1000 includes 1020 coupling a plurality of sensors 130 to the spastic upper limb 22 of the pediatric patient 20, wherein the sensors 130 are configured to track movement and orientation of the spastic upper limb 22 of the patient 30. The wearable device 100 allows the coupling of the sensors to be easily accomplished in the same manner as one attaches a watch. The coupled sensor can be tracking the movements and orientation data of the spastic limb 22 of the patient 20 that are relevant for the identified therapeutic movements.

### 1030 Transmitting

The method 1000 includes 1030 transmitting the tracked movement and orientation data of the spastic upper limb 22 of the pediatric patient 20 to a controller 200. Preferably this is accomplished in real time via a Bluetooth connection or similar data transfer protocol. The bracelet 100 can also include an onboard data storage device 150 that is removable such that the transmission is through the transfer of the removable data storage device 150 and coupling to the controller 200. Preferably the platform uses real time transmission of the data, and the onboard data storage 150 need not be removable and need only store the data until it is transmitted. The onboard data storage 150 can have sufficient capacity to store several days of data in account for when the bracelet is out of transmission range to the controller 200.

### 1040 Calculating

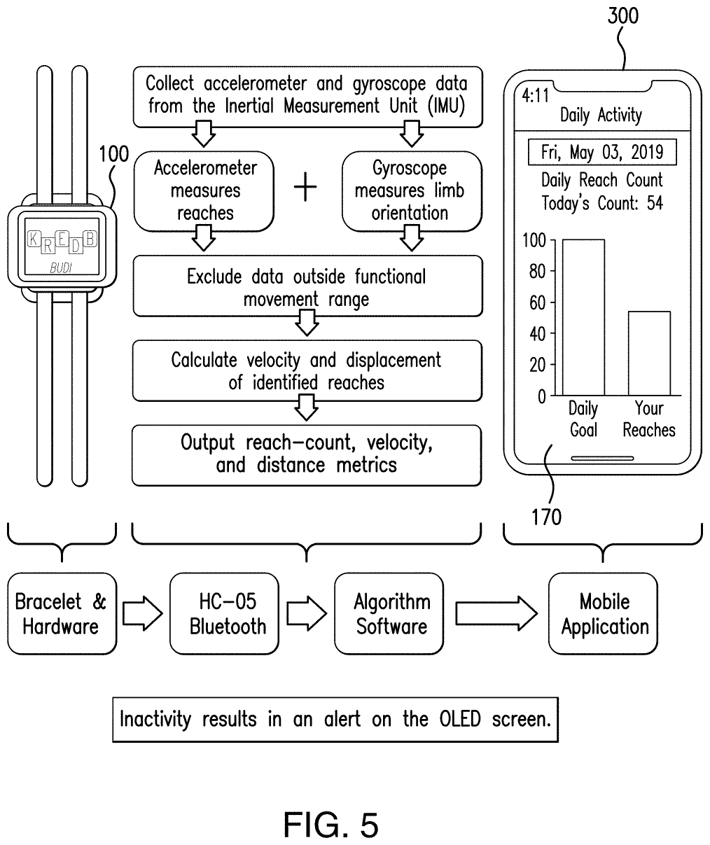

The method 1000 includes 1040 calculating a number of therapeutic movements of the spastic upper limb 22 of the patient 20 based upon the tracked movement and orientation data. As discussed above, for the forward reach as the desired motion, for example, the accelerometer data is primarily used to begin a count of reach using waveform recognition or detection and/or peak detection and the magnetometer and gyroscope data is used to generate exclusion criteria to eliminate “false positives” from the accelerometer data. FIG. 8 is a schematic chart of one process for calculating reach count for reach based therapeutic motion of the upper limb motion tracking biofeedback platform for children with hemiplegic spastic cerebral palsy according to FIG. 2 and FIG. 5 also schematically outlines the process used for calculating therapeutic reaches. The figures suggest using only the gyroscopic data for exclusion criteria, however, the magnetometer is also useful for identifying reasonable exclusion criteria however the method 1000 of rehabilitation therapy according to one aspect of the subject matter provides that the data obtained from the magnetometer is utilized to distinguish between incidental and therapeutic motion.

The particular algorithms used can vary, and the programming is known to those of ordinary skill in the art using IMU 130 for motion tracking, however, an algorithm is not considered acceptable unless it has greater than an 85% accuracy rate in counting therapeutic motions. The algorithm implemented can be sufficient such that involuntary background motion can not interfere with the ability to detect functional arm movements. For example in the forward reach motion on the platform 10, while the shaky reach (FIG. 6B) data proved to be much noisier than the normal forward reach (FIG. 6A), the overall pattern and shape for a forward reach was maintained. The linear acceleration data still showed the characteristic shape and amplitude of a forward reach on the x-axis when isolated from the other axis directions. The gyroscope and magnetometer data give further insight into the nature of the movement being performed and can be used to define exclusion criteria. It was clear from observing IMU x-acceleration data that characteristic waveforms appear for patient forward reaches of all speeds and directions allowing for waveform recognition methods, but the amplitudes of these waveforms vary from movement to movement and from person to person. Exclusion criteria are implemented in the algorithm, for example, the data has been filtered, centered, and zeroed, the pitch value at each data point is looked at to determine if this exceeds a threshold (i.e., 35 degrees).

### 150 Displaying

The method 1000 includes 1050 of displaying via one or more displays 170 at least the number of therapeutic movements of the spastic upper limb 22 to the patient 20 and/or the therapist 30. It is helpful if the information is graphically displayed to the patient 20 and in a manner that is easily understood by the patient. As the patients are children, there can be great care given to determining how best to convey the information in a positive yet informative manner. The method 1000 includes maintaining a historical record of therapy by the patient 20 and selectively displaying the same to the patient 20, clinician 30 or caregiver (possibly in different formats for each).

The method 1000 of rehabilitation therapy according to the present subject matter further including providing encouragement to the patient when falling behind a preset goal and accolades to the patient 20 when accomplishing a goal. Clinicians and caregivers can be notified of results and notifications of goals attained or a need to increase the pace of therapeutic motions.

The wearable device 100 collects the daily movement data of the affected arm 22 of a child 20 with HSCP and ultimately displays that data to the patient 20 (through the device 100 or a separate phone application on device 300) and send the data to their clinician 30 and possibly the caregivers.

Furthermore, platform 10 allows, where appropriate as determined by clinicians, caregivers and the individual user, this data to be shared with a community of the wearers or patients 20 of the present platform 10, again only with the consent of the patient 20 and/or parent and clinician 30. However, a concern that arises is one of privacy and consumer consent. This concern with privacy is widespread throughout wearable technology and health trackers. These devices have allowed the users to take more control of their health and are proving to be useful in reducing healthcare costs, but also serve as data collection tools. The respectively low cost of these devices and their integration with smartphones has caused the market to grow rapidly; however, the large adaptation of these devices allows for mass data collection, which can lead to discriminatory profiling, data breaches, and manipulative marketing. The platform 10 places the privacy of the patient 20 at the center of the equation when considering these concerns.

The Health Insurance Portability and Accountability Act (HIPAA) aims to ensure the safe of healthcare information. Even though there is a lot of ambiguity on whether HIPAA applies in the context of conventional wearable technology, in platform 10 it does due to the fact that the wearable device 100 of the platform 10 shares data with a clinician 30. The primary design constraint of the platform 10 is to only allow the data to be seen by the people who the wearer 20 (and caregivers) gives consent. This can address concerns of privacy and consumer consent and allows the platform to follow HIPAA guidelines, and further allows the system to easily adapt to any other privacy protocols developed in the future.

### The Platform 10 And Associated Method 1000 And Cerebral Palsy Therapy

A more detailed discussion of cerebral palsy is helpful for fully understanding the aspects and purposes and inherent advantages of the platform 10 and associated method 1000 of the preferred embodiment of the present subject matter.

### Pathophysiology

Cerebral palsy describes a group of non-progressive neuromuscular disorders that affect movement and posture. Most cases of cerebral palsy are thought to be caused prenatally, during birth, or immediately post-birth, due to exposure to radiation, infection, or hypoxic environments. Hypoxic environments are caused by the interruption of the oxygen supply from a mother to the fetus during gestation, birth, or directly post-birth. Brain asphyxia, or the lack of oxygen and blood to the developing brain, results in brain lesions due to damage that is done by a waste build-up in individual cells.

Infections during pregnancy are also risk factors for congenital cerebral palsy; the infections linked to congenital cerebral palsy can be chickenpox, rubella, and bacterial infections. These infections cause an increase in the level of inflammatory cytokines in the mother, which have been known to cause brain damage to the fetus.

The pathological brain injury seen in children 20 with cerebral palsy can be a periventricular white matter injury (PWMI) resulting from the vulnerability of immature oligodendrocytes before 32 weeks of gestation. This injury causes the spastic, diplegia form of cerebral palsy.

Brain lesions affect the nerves that control voluntary muscles and the nerves that communicate sensory information back to the brain. When the neurons become unhealthy or die, communication between the nervous system and muscles breaks down. As a result, muscles weaken and experience symptoms such as spasticity and stiffness. Spasticity means there is an increase in “muscle tone.” When the muscle is moved, there is more resistance to this movement, then there can normally be, and the muscles feel more rigid. Increased tone can mean muscles are slower to relax, and this can cause stiffness. Stiffness in the hands and arms makes it difficult to perform delicate movements while stiffness in the legs makes larger movements like walking more difficult.

Muscle in affected limbs 22 from patients 20 with cerebral palsy shows functional deficits such as decreased force production and range of motion. Contractures and spasticity in CP of affected limbs 22 can correspond to changes in muscle sarcomere length, fiber type, ECM concentration, fiber, and fiber bundle stiffness, and even stem cell numbers. The muscle of the affected limb 22 is also altered at a structural level, with decreased muscle body size, smaller-diameter fibers, and highly stretched sarcomeres (the force-producing unit of muscle). If typically developing sarcomeres are stretched, maximal force production can increase, whereas the opposite can happen for CP sarcomeres.

A child 20 with cerebral palsy can present deficits in expected developmental milestones. This can include failing to suppress obligatory primitive reflexes, such as the Moro startle reflex. Cerebral palsy often manifests as early hypertonia, and thus abnormal muscle tone is the most frequently observed symptom of cerebral palsy. There are three main types of cerebral palsy: spastic, dyskinetic, and ataxia. These types of cerebral palsy are segmented based on the area of the brain that is affected. Cerebral palsy can further be classified by the part of the body affected. The most severe case, quadriplegia, affects all four limbs and can further affect muscles in the face, trunk, and mouth. Diplegia affects both lower limbs, and the arms can be affected to a lesser extent. Finally, hemiplegia affects the arm 22 and leg on one side of the body. Thus, cerebral palsy encompasses a vast spectrum of movement disabilities, each of which has its own treatment demands.

Physical indicators of cerebral palsy include joint contractures due to spastic muscles, growth delay, and persistent primitive reflexes. Upon observation, this leads to asymmetry in human movement and control, including asymmetric gait, posture, strength, and coordination. Affected upper limbs 22 can present as abnormal contracture at the elbow, and the wrist and children 20 with CP have a difficult time controlling their motor functions. These joint contractures cause a limited range of motion in the affected limb 22.

Symptoms in the lower limbs can present as gait abnormalities. There are several well-characterized types of gait, including scissor gait, toe walking, and crouch gait. Scissor gait is characterized by hypertonia in the legs and hips and extreme adduction causing the thighs to hit or cross one another in a “scissor-like” fashion. Toe walking is characterized by toe-to-toe bilateral gait; it is considered a normal part of gait development; however, it becomes abnormal once past the age of 2. Finally, crouch gait is characterized by excessive dorsiflexion of the ankle and flexion at the knees and hip causing one to be in a “crouched” position while walking.

The Gross Motor Function Classification System (GMFCS) is a standard scale used to classify the movement ability of a child 20. It accurately predicts the functional capabilities of children 20 with cerebral palsy. There are five levels that range from Level I, walking with limitation and good coordination, to Level V, being completely paralyzed and wheelchair bound. Those in GMFCS Level I can perform many motor tasks, such as running and jumping, but their coordination is impaired. Those in GMFCS Level II can walk and climb stairs (with support from the railing), but experience difficulty walking on uneven surfaces or in confined spaces. Those in GMFCS Level III are able to walk with an assistive device and are able to operate a wheelchair by themselves. Those in GMFCS Level IV can walk short distances with an assistive device but are more reliant on wheeled transportation. Lastly, those in GMFCS Level V can not be able to control their trunk posture and are not capable of independent mobility.

### Clinical Outcomes

Cerebral palsy manifests muscular impairments in children 20, resulting in poor motor control and impairments in gait, balance, posture, and coordination. Spastic hemiplegia in cerebral palsy means that affected patients 20 have one side of the body with stiffer, less-coordinated muscles than the other. There is a tendency for children 20 to neglect their affected upper limb 22 in their daily life. Neglect of the affected upper limb 22 and compensating by performing unilateral tasks with the unaffected upper limb 24 can lead to both physical and psychological/social clinical consequences. While cerebral palsy is a non-progressive disorder, with high neural and muscular plasticity at a younger age, children 20 with cerebral palsy are at risk of muscular degeneration that can ultimately reclassify them at a higher level (more severe case) on the GMFCS base on muscular and motor function. About 40% of children 20 can be reclassified one or two levels over time, and further revealed that children 20 with unilateral involvement (hemiplegia) were more often reclassified by two levels than children with bilateral involvement.

On the physical consequences, upper limb neglect can lead to poor muscular development, muscle weakness, poor range of motion, poor flexibility, and muscle degeneration.

On the range of motion in cerebral palsy, people 20 with cerebral palsy are more likely to have a decreased range of motion (ROM) because of reduced mobility and the presence of spasticity and dystonia. Having adequate muscle and joint movement allows a person to move freely and efficiently and is an important part of completing everyday activities for everyone, including people 20 with cerebral palsy. Reduced range of motion can affect a person's ability to carry out everyday activities and can lead to secondary problems such as strain and discomfort, contractures, and hip displacement and dislocation.

On the psychological and social consequences, children 20 with hemiplegic cerebral palsy can be in the subgroup of individuals with cerebral palsy that is more likely to complete mainstream schooling and obtain gainful employment. However, the physical, and sometimes cognitive, difficulties associated with hemiplegic cerebral palsy can put these individuals 20 at a disadvantage in the mainstream environment and thus impact their self-esteem and quality of life.

Certain children with cerebral palsy can have low self-esteem. The level of self-esteem can be verified through a comprehensive quantification of self-concept and quality of life in children with cerebral palsy and age-matched, healthy individuals. Children 20 with cerebral palsy can have lower scores on self-esteem and self-confidence, reduced self-concept, and lower quality of life than typically developing peers.

Relating to the psychological consequences, the behavior of children 20 with cerebral palsy can be described with the term “developmental disregard.” Developmental disregard refers to a discrepancy between capacity and performance in these individuals 20. This means that there is a gap between what a child 20 thinks that he or she can do and what he or she can actually do. This is particularly important for the affected limbs 22 of children 20 with hemiplegia. The developmental disregard suggests that the physical and psychological consequences outlined for hemiplegic cerebral palsy are connected and can build on each other in a vicious cycle.

The physical and developmental clinical consequences associated with cerebral palsy ultimately can restrict the daily activities that these children 20 can perform independently. The clinical outcomes of children 20 with these impairments require continuous therapy throughout the child's development.

### Epidemiology

Cerebral palsy is a motor disability in children. According to the Centers for Disease Control and Prevention, world population-based studies estimate that 1.5-4 per 1000 live births result in the onset of cerebral palsy and that 17 million people are affected with the disorder overall. The CDC's Autism and Developmental Disabilities Monitoring Network estimates that 1 in 323 children have been identified with cerebral palsy in 2017. Spastic is a motor type of cerebral palsy, affecting 70-80% of diagnosed individuals. Dyskinetic and ataxic each make up an additional 6% while the remaining individuals suffer from combination damage.

There is an even distribution among cerebral palsy cases between the affected body regions with 39% of patients being hemiplegic, 38% of patients being diplegic, and the remaining 23% of patients are quadriplegic. More than half of children diagnosed with cerebral palsy can walk independently while 10% walk with some form of aid. The remaining 30% are wheelchair bound, requiring constant attendance. 83% of children with cerebral palsy have upper limb impairments, 69% have reduced hand control, and 36% have demonstrated contracture at the elbow and/or wrist. In total, children 20 with hemiplegic spastic cerebral palsy make up 22.6% of diagnosed children.

### Economic Impact

Cerebral palsy is a long-term chronic medical condition that subsequently requires longterm supportive care services. People with cerebral palsy incur an average total annual cost of $11.5 billion amongst the associated medical, scientific, educational, and research communities. The continuous therapy required by children 20 with cerebral palsy is a heavy financial burden on the family. For an individual family, a lifetime cost of $921,000 per person with cerebral palsy is estimated. This estimate is comprised of 80.6% indirect costs, 10.2% direct medical costs, and 9.2% direct non-medical costs. Direct medical costs include physician's visits, prescription medications, and therapies. Direct non-medical costs include special education and home and automobile modifications. Indirect costs include the inability to work and premature mortality. Additionally, among 18 common congenital disorders, which means a condition that is present at birth, cerebral palsy has the highest associated lifetime cost.

### Treatment Overview

There is no cure for cerebral palsy. A range of treatments are available and are continuously implemented through the development of a child 20 to help treat as many of the symptoms of cerebral palsy as possible and to enable children 20 to be as independent as possible. These treatments include rehabilitation therapy (physical therapy and occupational therapy). Physical therapy is a means of improving muscle tone and movement patterns, with the main goals of preventing the weakening of muscles that are not normally used by the child, preventing muscle shortening and losing normal range of motion, and helping move as independently as possible.

Rehabilitation therapy is carried out by a licensed therapist 30, and he or she guides the child 20 through the exercises. Again in the context of this application, therapist 30 and clinician 30 are used interchangeably as a care provider, as each interacts with the platform 10 in the same manner, but in the broader context, the terms reference distinct individuals. Certain methods of rehabilitation therapy for the patient 20 include soft tissue mobilization, which is carried out by kneading the muscles, joint mobilization, specialized exercises, stretching, and endurance exercises. Additionally, these exercises often include equipment such as weights, exercise machines, bands, rollers, balance balls, heat and cold packs, and ultrasound technology. While these are certain methods used in rehabilitation therapy, it is a very individualized process, and therapists create individual exercise programs to account for each child's strengths and weaknesses.

The overall goal of occupational therapy is to develop a child's ability to perform daily activities that improve their quality of life and enable them to be independent. This type of therapy typically involves carrying out everyday tasks and finding solutions. Some of these tasks include home activities including eating, bathing, and using a computer; school activities such as opening a locker and holding books and supplies; and community activities such as navigating public spaces. The purpose of occupational therapy is to teach children 20 with CP how to think critically, make decisions, and solve problems based on the task put in front of them. An important part of this involves breaking down these tasks into smaller, more doable tasks that allow the child 20 to perform and succeed. Since occupational therapy is focused on developing skills for everyday tasks, a wide range of equipment is used, including games, toys, household objects, and supplies such as pencils and scissors.

Certain occupational therapy for upper limb impairments can be Constraint-Induced Movement Therapy (CIMT), where the clinician 20 restricts the movement of the unaffected arm 24 to enforce the use of the affected arm 22. The restraint can be applied for anywhere from a few hours up to 24 hours each day for 2 to 4 weeks. This type of therapy has been successful in children 20 with hemiplegic cerebral palsy, showing the improved quality of hand use, new and more spontaneous motor movements of the affected arm and hand, and improved hand function such as fine motor and grasp.

Certain techniques can be used to assess the effectiveness of Constraint-Induced Movement Therapy in children 20 between the ages of one and five years old by comparing a 4-week baseline period with no treatment and a second 4-week treatment period that consisted of twice-weekly 1-hour sessions and a home program for non-treatment days. The therapy can show improvements in the affected arm 22. These improvements can be quantified through the Quality of Upper Extremity Skills Test (QUEST), which evaluates the quality of upper limb function through four domains: dissociated movement, grasps, protective extension, and weight bearing. After the 4-week treatment period, the average QUEST score increased. CIMT has also shown improvements to the psychosocial clinical consequences discussed earlier, such as lack of self-confidence and dependence on others.

Another effect of CIMT includes motor cortex neural plasticity. Increases in the portion of the motor cortex responsible for controlling the thumb's abductor pollicis brevis (APB) muscle following CIMT. The APB muscle is used in the hand during grasping, which is an exercise often trained during CIMT. These motor cortex improvements can be also associated with improvements to the functional ability of the affected upper limb. The improvements can be quantified through the Motor Activity Log (MAL), which consists of 20 tasks that reflect activities carried out in daily living such as feeding, grooming, and dressing. These increases in the MAL can suggest that the affected arm 22 was used more in everyday life after treatment.

Another more recently developed occupational therapy for children with hemiplegic cerebral palsy is Bilateral Movement Therapy, or Hand-Arm Bimanual Intensive Therapy (HABIT). It is similar to CIMT, however, the less affected arm is not restrained in a sling or a cast, which allows for bimanual training. HABIT aims to improve the use and coordination of both arms in coordination in daily function. Like CIMT, it requires 90 hours of intensive therapy and it is performed in group settings with an emphasis on providing a fun environment for the children 20. HABIT can be equally as effective as Constraint-Induced Movement Therapy for patients 20. the Jebsen-Taylor Test of Hand Function, which evaluates fine and gross motor hand function using activities of daily living, and Assisting Hand Assessment, which measures how effective the affected hand and arm (limb 22) is during bimanual tasks, can be used to show score increases following both treatments, leading to the conclusion that the efficacy of affected arm rehabilitation is not dependent on the use of restrictive devices on the unaffected arm 24.

Botulinum toxin treatment is used and is known to reduce spasticity and increase the range of movement in a joint, which can ultimately help with therapy. However, this treatment only works short-term and requires frequent administration. Additionally, it is thought that in the long-term, botulinum toxin can result in muscle weakness and atrophy, so its use is not a common practice. Additionally, there are a few surgical treatments for the affected limb 22 which include tendon transfer, muscle lengthening, and arthrodesis, or surgical immobilization of the joint.

Gap Analysis—Fit of Platform 10 into CP Treatments

The above review of the Pathophysiology, Clinical Outcomes, Epidemiology, Economic Impact and Treatment Overviews of children 20 with Cerebral Palsy help clarify the operation and purpose of the platform 10 and associated method 1000 of the present subject matter in complementing and supplementing existing treatments, while allowing for the potential to develop new treatments incorporating the platform 10 of the present subject matter. Within the cycle of care for cerebral palsy in children 20, the platform 10 and method 1000 of the subject matter addresses the at-home care of these children 20. This falls between treatment and follow-up, both which occur in the presence of a clinician 30. It is important to address this step in the cycle of care because children 20 spend hours in treatment with clinicians 30, such as physical and occupational therapists, and see positive results in the affected upper limb 22 from this therapy, but there is a drop off or loss between therapy sessions. The platform 10 and method 1000 encourages children 20 with hemiplegic spastic cerebral palsy to use their affected upper limb 22 in their everyday life outside of the presence of the clinician 30.

As discussed above, physical therapists and occupational therapists 30 often implement Constraint-Induced Movement Therapy in children 20 with hemiplegic cerebral palsy and this has shown a localized increase in gray matter volume of the sensorimotor cortex contralateral to the affected arm 22 targeted during rehabilitation.

Improvement of capacity and performance of the upper limb through Constraint-Induced Movement Therapy followed by Bimanual Training (mCIMT-BiT) in children 20 with unilateral spastic CP is based on better utilization of existing motor functions of the affected limb 22.

These training method can produce limited long-term movement improvements, as the movements learned in training are rarely actively continued outside of the clinic. As follow-up time after therapy increased, the effect of therapy decreased. This shows that Constraint-Induced Movement Therapy cannot be maintained over time due to the fact that children 20 do not practice with their affected arm 22 as much after therapy. The effect of the newly learned function from therapy can fade away without proper practice and reminding. Another downfall of Constraint-Induced Movement Therapy is that the child 20 is at an increased risk of falls and loss of balance since one arm 24 is constrained and can affect posture and the ability to prevent oneself from falling.

Thus, there is an opportunity for an intervention platform 10 of the subject matter that continually encourages upper limb 22 usage of hemiplegic cerebral palsy patients' affected limbs 22 to fulfill the role of the clinician or therapist 30 outside of the clinic. The platform 10 is designed to provide a fun game-like aspect for the child. Children 20 with neurodevelopmental disorders like cerebral palsy are in need of fun and engaging rehabilitation methods to enhance motivation and increase compliance with motor training, such as the video game. The game can make therapy a positive experience and enable the children 20 to be more independent in their therapy, which can be resulted in a lower need for parents to constantly monitor and coach their children 20. Additionally, the fact that the game had a social aspect since they can play with other people was also positive for the children 20, and the platform 10 can similarly incorporate social aspects into its implementation to yield similar benefits.

The platform 10 yields, in one aspect of the subject matter, a reminder system and progress feedback system or compliance system for the patient 20, and the efficacy of reminder systems and progress feedback systems is supported by the scientific research. The reminder systems and progress feedback have the greatest impact on compliance, achievement outcomes, and maintenance. This is a characteristic of the present platform 10 wherein by reminding children 20 with hemiplegic, spastic cerebral palsy to use their affected upper limb 22 in their daily life, there is a higher likelihood of better muscular, kinetic, and biomechanical bilateral symmetry in the upper limbs 22 and 24.

The platform 10 of the present subject matter has been designed with a number of functional guidelines to assure adoption and effective use by the patients 20 and their caregivers. The first is that the quantification of upper limb usage can be meaningful, namely that the sensitivity, specificity, accuracy and precision of the quantification of the upper limb usage 22 can be at least 85%. The platform 10 exceeds this quantification requirement. The platform 10 is capable of real time or near real time display of therapeutic motion, which for a reach as a therapeutic motion can include the reach count, as well as distance and velocity (assuming the latter two criteria are meaningful). The platform 10 is designed to encourage children 20 to actively use their affected limb 22 in daily life to maintain post treatment function and activity levels achieved in therapy. The platform 10 is designed to be non-restrictive to daily tasks in that the wearable subassembly 100 weighs less than 48 grams and is of a size comparable to other wearable devices, like watches, or the APPLE® watch, or FITBIT fitness tracking devices. The platform 10 is safe to use and includes appropriate data encryption and is formed of biocompatible materials. The wearable subassembly 100 is user friendly for children 20 with CP by being easy to put on and take off and is cosmetically appealing to the children 20. The platform follows the above functional guidelines to result in a platform 10 that allows for wide adoption by the relevant user base and is effective at quantifying upper limb usage, encouraging children 20 to use their affected upper limb in the absence of a clinician 30, and discouraging neglect of the affected upper limb 22 during bilateral tasks. The platform 10 attempts to quantify upper limb usage in order to provide optimal encouragement and reminding, and it also documents upper limb usage so that parents, clinicians 30, and the children 20 can view progress.

The above functional guidelines set the platform 10 apart of the current gold standard treatment, CIMT. For example, the platform 10 is not restrictive to any daily tasks that the child 20 can perform and is much safer for the child 20 particularly in the non-clinical environment. The non-restrictive device of the wearable subassembly 100 of the platform 10 is simply safer than having one arm fully restricted or casted. The platform 10 is user-friendly for children 10 with cerebral palsy allowing it to form an effective tool. Anything too complicated can discourage the child 20 from using it. Finally, the present functional guidelines can match those of other movement tracking wearable devices with regard to usability.

### First Supplemental or Alternative Wearable Device

A first alternative or first supplemental embodiment of the present subject matter is an activity measurement arm sleeve that can be in place of, or possibly supplemental to, the wearable device 100. It can work with the remaining aspects of the platform 10 as discussed above. The second embodiment includes a wearable device formed as a pair of arm sleeves that include electromyographs to quantify muscle activity, electrogoniometers to monitor joint angles, and accelerometers to monitor wrist kinetics. Electromyographs (EMGs) use intramuscular electrodes, or electrodes placed on the skin, to measure the electrical activity of muscles. Skeletal muscle usually does not produce electrical signals during rest; therefore, with the EMGs, the amount of activity versus the amount of rest can be seen. Additionally, there is a correlation between the amount of muscular electrical activity and strength of muscular contraction; therefore, the EMG can measure the extent in which the child 20 uses both the affected arm 22 and the unaffected arm 24. Similarly, an electrogoniometer is a tool that allows for the continuous measurement of joint angles. Due to the fact that children 20 with spastic hemiplegic cerebral palsy can develop joint contractures in their upper limb 22 (which results in a limited range of mobility), the electrogoniometer can measure the change in angle of their elbow joint and quantify the movement from this contracted position. Lastly, an accelerometer measures acceleration based on the force needed for movement. Many accelerometers use piezo crystals that generate an electrical signal that is proportional to the force acting on the crystal. The accelerometer can detect the start of movements and the speed of movements of the limbs. Together, the EMG, electrogoniometer, and accelerometer can paint a clear picture of the movement of the arms. These sleeves can communicate with each other and provide biofeedback on arm usage, indicating when the ratio of affected arm 22 to unaffected arm 24 usage is too low. Additionally, the sleeve can signal to the child 20 when the affected arm 22 usage is too low, can be light up or vibrate so that the child 20 knows they can start to use the affected arm 22 more.

### Second Supplemental or Alternative Wearable Device

A second alternative or second supplemental embodiment of the present subject matter is a pair of pressure-sensing gloves that can be in place of, or possibly supplemental to, the wearable device 100. The gloves can work with the remaining aspects of the platform 10 as discussed above.

These gloves can contain piezoelectric crystal materials to quantify hand usage. When a pressure is applied to a piezoelectric crystal, it generates a charge. Typically, piezoelectric pressure sensor systems output an AC coupled signal. The operation of such devices is outlined in PCB Piezotronics, “Introduction to Dynamics pressure sensors,” PCB Piezotronics MTS Systems Corporation. When the child wearing the glove touches something, the piezoelectric crystals can deform, generating a charge. This charge can then travel through wires from the tips of the fingers to the palm of the hand to the base of the glove around the wrist, where it can be collected and stored. As in the previous supplemental device, the gloves can be able to communicate with each other to monitor how much the affected arm 22 is being used compared to the unaffected arm 24. As data is being collected, the gloves can further provide feedback in the form of lights when affected arm use is too low.

Alternative Therapeutic Motions and or affected limbs or conditions

The platform 10 and associated method 1000 of the present subject matter has been defined in particular in accordance with reach for therapeutic motion in the method of motion tracking, biofeedback based, upper limb rehabilitation therapy for a pediatric patient 20 with hemiplegic spastic cerebral palsy. Other motions than reach can be used with the platform 10. For example the therapist can desire the patient reach vertically overhead with the affected arm. As discussed above, the desired motion is modeled and the sensor can detect inputs to reliably identify the desired therapeutic motion. As noted above—reliability is greater than 85% accuracy within the meaning of this application. The platform can simply track all of a series of known therapeutic motions with the feedback based upon a desired amounts based upon the therapist 30 guidelines specific to the patient. It is possible the platform 10 includes a drop-down screen in which the clinician 30 selects the desired motions to be tracked and the unselected motions are simply not displayed.

The platform 10 and method 1000 as disclosed can be equally applicable to patients that are experiencing spasticity in one upper limb 22 even if not caused by CP. CP is simply the most likely cause for such a diagnosis.

The platform 10 and associated method 1000 discussed above can be easily adapted for gait training and for use on affected lower limbs, with stride length and timing and foot position being relevant parameters to be considered with the platform.

Although the present invention has been described with particularity herein, the scope of the present invention is not limited to the specific embodiment disclosed. It will be apparent to those of ordinary skill in the art that various modifications can be made to the present invention without departing from the spirit and scope thereof. The scope of the present invention should be defined by the appended claims and equivalents thereto.

What is claimed is:

1. A method of motion tracking, biofeedback based, limb rehabilitation therapy for a patient comprising:
defining a desired therapeutic motion for a limb of the patient;
coupling at least one sensor to the limb of the patient wherein the sensor is configured to track at least one of movement and orientation of the limb;
transmitting the tracked movement and/or orientation data to a controller;
calculating a number of therapeutic movements of the limb of the patient based upon the tracked movement and/or orientation data;
displaying at least the number of therapeutic movements to the patient.

2. The method of claim 1, further including setting at least one goal for the patient and providing positive reinforcement to the patient upon attaining a preset goal.

3. The method of claim 1, further including maintaining a historical record of therapy by the patient.

4. The method of claim 1, further including providing encouragement to the patient when falling behind a preset goal.

5. The method of claim 1, further including graphically displaying the data to the patient.

6. The method of claim 1, wherein the limb is an upper limb of the patient and wherein the coupling at least one sensor to the limb of the patient includes coupling at least one sensor to the wrist of the user.

7. The method of claim 1, wherein the calculating a number of therapeutic movements of the limb of the patient based upon the tracked movement and/or orientation data includes waveform recognition.

8. The method of claim 1, further including maintaining a record of clinician review of the data.

9. A system for motion tracking, biofeedback based, upper limb rehabilitation therapy for a pediatric patient with hemiplegic spastic cerebral palsy, comprising:
one or more processors configured to
define a desired therapeutic motion for a spastic upper limb of the pediatric patient;
couple a plurality of sensors to the spastic upper limb of the pediatric patient, wherein the sensors are configured to track movement and orientation of the spastic upper limb;
analyze the tracked movement and orientation data of the spastic upper limb of the pediatric patient;
calculate a number of therapeutic movements of the spastic upper limb of the patient based upon the tracked movement and orientation data;
display at least the number of therapeutic movements of the spastic upper limb to the patient.

10. The system of claim 9, wherein the plurality of sensors include at least one of a magnetometer, an accelerometer, or a gyroscope, wherein data obtained from the plurality of sensors is utilized to distinguish between incidental and therapeutic motion.

11. The system of claim 10, wherein the processor is configured to calculate the number of therapeutic movements of the spastic upper limb of the pediatric patient based a waveform recognition.

12. A limb motion tracking biofeedback platform comprising:
a plurality of sensors configured to be coupled to a limb of a patient, wherein the sensors are configured to track and transmit movement and orientation of the limb;
one or more processors configured to calculate a number of predetermined therapeutic movements of the limb based upon the tracked movement and orientation data; and
a display configured to display at least the number of therapeutic movements of the limb to the patient.

13. The limb motion tracking biofeedback platform of claim 12, wherein the limb comprises a spastic upper limb.

14. The limb motion tracking biofeedback platform of claim 12, wherein the patient comprises a pediatric patient.

15. The limb motion tracking biofeedback platform of claim 12, wherein the plurality of sensors are configured to be coupled to a wrist of the patient.

16. The limb motion tracking biofeedback platform of claim 12, wherein the limb motion tracking biofeedback platform is in a form of a watch.

17. The limb motion tracking biofeedback platform of claim 12, wherein the plurality of sensors can comprise at least one of a magnetometer, an accelerometer, a gyroscope, or combinations thereof

18. The limb motion tracking biofeedback platform of claim 12, wherein the tracked movement and/or orientation data comprises waveform data measured from the patient.

19. The limb motion tracking biofeedback platform of claim 12, wherein the magnetometer is configured to generate data for distinguishing incidental and therapeutic motion.

20. The limb motion tracking biofeedback platform of claim 12, wherein the one or more processors configured to
define a desired therapeutic motion;
couple a plurality of sensors to the limb of the patient;
analyze the tracked movement and orientation data of the spastic upper limb of the pediatric patient to a controller;
calculate a number of therapeutic movements of the spastic upper limb of the patient based upon the tracked movement and orientation data; and
display at least the number of therapeutic movements through the display.
